(19) United States
(12) Patent Application Publication
(10) Pub. No.: US 2019/0136191 A1
(43) Pub. Date: May 9, 2019

(54) Methods of Producing Specialized Cardio-Like Cells from Stem Cells

(71) Applicants: Emory University, Atlanta, GA (US); Children's Healthcare of Atlanta, Inc., Atlanta, GA (US)

(72) Inventor: Hee Cheol Cho, Johns Creek, GA (US)

(21) Appl. No.: 16/098,653

(22) PCT Filed: May 2, 2017
(86) PCT No.: PCT/US2017/030665

§ 371 Date: Nov. 2, 2018

Related U.S. Application Data
(60) Provisional application No. 62/420,904, filed on Nov. 11, 2016.
(60) Provisional application No. 62/330,506, filed on May 2, 2016.

(51) Int. Cl. C12N 5/077; C12N 5/0735

(52) U.S. Cl. CPC C12N 5/0657; C12N 5/0606; C12N 2501/385; C12N 2501/155; C12N 2501/16; C12N 2501/115

(57) ABSTRACT

This disclosure relates to method of differentiating stem cells to specific cardiac-like cells. In certain embodiments, the disclosure contemplates methods of generating left ventricular-like cells and the atrial-like cells by timing the exposure of dividing stem cells to retinoic acid (RA) or retinoic acid receptor inhibitors.

### CROSS REFERENCE TO RELATED APPLICATIONS

This application claims the benefit of U.S. Provisional Application No. 62/330,506 filed May 2, 2016 and U.S. Provisional Application No. 62/420,904 filed Nov. 11, 2016. The entirety of each of these applications is hereby incorporated by reference for all purposes.

### STATEMENT REGARDING FEDERALLY FUNDED RESEARCH

This invention was made with government support under 5R01 HL 111646-05 awarded by the National Institutes of Health. The government has certain rights in the invention.

### BACKGROUND

Cardiovascular disease (CVD) is capable of irreversibly damaging heart cells. Stem cells are a promising therapeutic for restoring damaged heart muscle tissue. Embryonic and pluripotent stem cells can give rise to many different organ tissues, including the heart. Cardiomyocytes in the atrial and ventricular chambers of the heart are distinct and express unique cell marker patterns. However, cardiac myocytes derived from stem cells using known methods are typically heterogeneous mixtures. Thus, there is a need to identify improved methods of generating more specialized cardiac cells.

Wobus et al. report retinoic acid accelerates embryonic stem cell-derived cardiac differentiation and enhances development of ventricular cardiomyocytes. J Mol Cell Cardiol. 1997, 29(6):1525-39.

Devalla et al. report modulating retinoic acid signaling during hESC differentiation can generate atrial-like (hESC-atrial) and ventricular-like (hESC-ventricular) cardiomyocytes. EMBO Mol Med. 2015, 7(4):394-410. See also Zhang et al., Cell Res. 2011, 21(4):579-87; U.S. Pat. Nos. 7,498,171, 6,887,704, 5,190,876 and U.S. Patent Application Publication Nos. 2003/0032183 and 2002/0142457.

References cited herein are not an admission of prior art.

### SUMMARY

This disclosure relates to method of differentiating stem cells to specific cardiac-like cells. In certain embodiments, the disclosure contemplates methods of generating left ventricular-like cells and the atrial-like cells by timing the exposure of dividing stem cells to retinoic acid (RA) or retinoic acid receptor inhibitors.

In certain embodiments, the disclosure contemplates inhibition of the RA signaling during early differentiation followed by activation of the RA signaling during late differentiation leading to a predominantly left ventricular-like cell population. Atrial-like cells can be derived by activating the RA signaling during late differentiation with no early inhibition.

In certain embodiments, the disclosure relates to methods of making ventricular-like cells comprising: a) mixing stem cells with Activin A, BMP4, bFGF, and an inhibitor of retinoic acid signaling providing inhibitor treated dividing cells; b) mixing the inhibitor treated dividing cells with noggin providing noggin treated cells; and c) mixing the noggin treated cells with retinoic acid and a Wnt inhibitor providing ventricular-like cells. In certain embodiments, the ventricular-like cells are positive for Nkx2.5, are positive for IRX4, are positive for HRT2, and are negative for ISL1. In certain embodiments, the inhibitor treated dividing cells are exposed to the inhibitor of retinoic acid signaling for not more than one day.

In certain embodiments, the inhibitor treated dividing cells are exposed to Activin A, BMP4, and bFGF for not more than three days. In certain embodiments, the noggin treated cells are exposed to noggin for not more than two days. In certain embodiments, the mixing the noggin treated cells with retinoic acid is started five days after mixing stem cells with Activin A, BMP4, bFGF, and an inhibitor of retinoic acid signaling. In certain embodiments, the noggin treated cells are exposed to retinoic acid for not more than three days. In certain embodiments, the mixing the noggin treated cells with a Wnt inhibitor is started five days after mixing stem cells with Activin A, BMP4, bFGF, and an inhibitor of retinoic acid signaling. In certain embodiments, the noggin treated cells are exposed to the Wnt inhibitor for not more than six days.

In certain embodiment, this disclosure relates to methods of making atrial-like cells comprising: a) mixing stem cells with Activin A, BMP4, and bFGF providing treated dividing cells; b) mixing the treated dividing cells with noggin providing noggin treated cells; and c) mixing the noggin treated cells with retinoic acid and a Wnt inhibitor providing atrial-like cells. In certain embodiment, the atrial-like cells are positive for Nkx2.5, are positive for Coup TF II, are negative for IRX4, and are negative for ISL1. In certain embodiment, the treated dividing cells are exposed to Activin A, BMP4, and bFGF for not more than three days. In certain embodiment, the noggin treated cells are exposed to noggin for not more than two days. In certain embodiment, the noggin treated cells with retinoic acid is started five days after mixing stem cells with Activin A, BMP4, and bFGF. In certain embodiment, the noggin treated cells are exposed to retinoic acid for not more than three days. In certain embodiment, mixing the noggin treated cells with a Wnt inhibitor is started five days after mixing stem cells with Activin A, BMP4, and bFGF. In certain embodiment, the noggin treated cells are exposed to the Wnt inhibitor for not more than six days.

In certain embodiment, this disclosure relates to methods of making ventricular-like cells comprising: a) mixing stem cells with Activin A, BMP4, and bFGF providing treated dividing cells; b) mixing the treated dividing cells with noggin providing noggin treated cells; and c) mixing the noggin treated cells with a Wnt inhibitor providing ventricular-like cells. In certain embodiment, ventricular-like cells are positive for Nkx2.5, are positive for IRX4, and are positive for ISL1. In certain embodiment, the treated dividing cells are exposed to Activin A, BMP4, and bFGF for not more than three days. In certain embodiment, the noggin treated cells are exposed to noggin for not more than two days. In certain embodiment, mixing the noggin treated cells with a Wnt inhibitor is started five days after mixing stem cells with Activin A, BMP4, and bFGF. In certain embodiment, the noggin treated cells are exposed to the Wnt inhibitor for not more than six days.

In certain embodiments, the inhibitor of retinoic acid signaling is (E)-4-(2-(5,5-dimethyl-8-(phenylethynyl)-5,6-dihydronaphthalen-2-yl)vinyl)benzoic acid (BMS493), derivative, or salt thereof.

In certain embodiments, the Wnt inhibitor is 2-(4-(2-methylpyridin-4-yl)phenyl)-N-(4-(pyridin-3-yl)phenyl)acetamide (Wnt-059), derivatives, or salts thereof.

In certain embodiments, the stem cell is an embryonic stem cell, pluripotent stem cell, induced pluripotent cell, mesenchymal stromal cell, mesenchymal stem cell, and adipose tissue-derived multipotent stem cell.

In certain embodiments, the disclosure contemplates methods of testing or screening compound libraries for desirable physical properties comprising the steps of mixing left ventricular-like cells, atrial-like cells, or ventricular-like cells as described herein and a test compound, and determining, measuring, observing, recording on a computer readable medium, or transmitting through electronic means the data or effect of the text compound on a property of the cell. In certain embodiments, the property of the cells is selected from an abnormal impulse initiation, or abnormal conduction, automaticity, early (EAD) or delayed (DAD) afterdepolarizations, a long or short excitable gap, target ion current, effective refractory period, decreasing excitability, block of conduction K+, Na+, Ca2+ channel, a short excitable gap, prolong the action potential, a long excitable gap, agonism or antagonism of cardiac ion channels, pumps and receptors.

In certain embodiments, the disclosure contemplates a growth medium composition comprising cells and agents disclosed herein. Thus, in certain embodiments, this disclosure relates to a growth media comprising a stem cell, Activin A, BMP4, and bFGF. In certain embodiments, the growth media further comprises noggin to provide a noggin containing media. In certain embodiments, the noggin containing media further comprises a Wnt inhibitor disclosed herein providing a Wnt inhibitor containing media. In certain embodiments, the Wnt inhibitor containing media comprises retinoic acid.

In certain embodiments, this disclosure relates to a growth media comprising an inhibitor of retinoic acid signaling disclosed herein, a stem cell, Activin A, BMP4, and bFGF. In certain embodiments, the growth media further comprises noggin to provide a noggin containing media. In certain embodiments, the noggin containing media further comprises a Wnt inhibitor disclosed herein providing a Wnt inhibitor containing media. In certain embodiments, the Wnt inhibitor containing media comprises retinoic acid.

### BRIEF DESCRIPTION OF THE DRAWINGS

FIG. 1 illustrates a method of producing specific cardiac-like cells from stem cells. RA is retinoic acid, RA inhibitor (Ri) is retinoic acid inhibitor; D-3 to D14 are three days before and till 14 days after the start of cardiac differentiation; ActivinA, bFGF, BMP4 (AFB) are recombinant proteins that are needed at the start of cardiac differentiation; Noggin is a secreted protein that promotes somite patterning in developing embryo; C59 is an inhibitor of Wnt signaling.

FIG. 2A shows data for the IRX4 marker using human embryonic stem cells in the Ri+AFB+RA protocol outlined in FIG. 1. IRX4 and HRT2 are ventricular marker. CoupTFII is an atrial marker. NKX2.5, TBX5, HCN4 are markers of the first heart field, which becomes the left ventricular and atrial cardiac myocytes.

FIG. 2B shows data for the Hrt2 marker.

FIG. 2C shows data for the Coup TF II marker.

FIG. 2D shows data for the HCN4 marker.

FIG. 2E shows data for the Nkx2.5 marker.

FIG. 2F shows data for the Tbx5 marker.

FIG. 3A shows data for the ISL1 maker using the AFB in FIG. 1 without Ri and without RA. Is11, Mef2c, Fgf8, and Fgf10 are markers of the second heart field, which become right ventricular cardiac myocytes.

FIG. 3B shows data for the Mef2c marker.

FIG. 3C shows data for the FGF8 marker.

FIG. 3D shows data for the FGF10 marker.

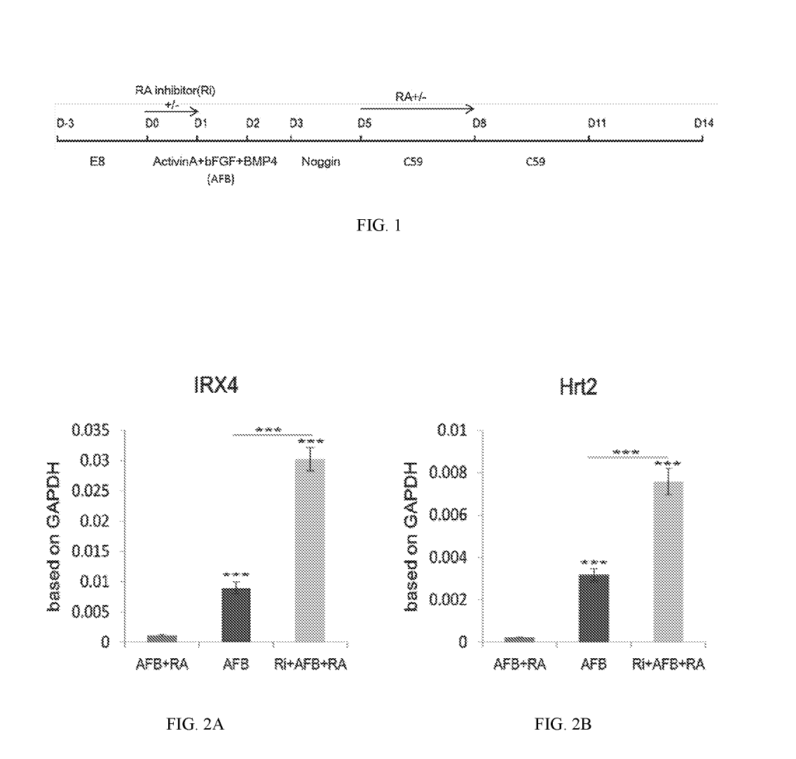

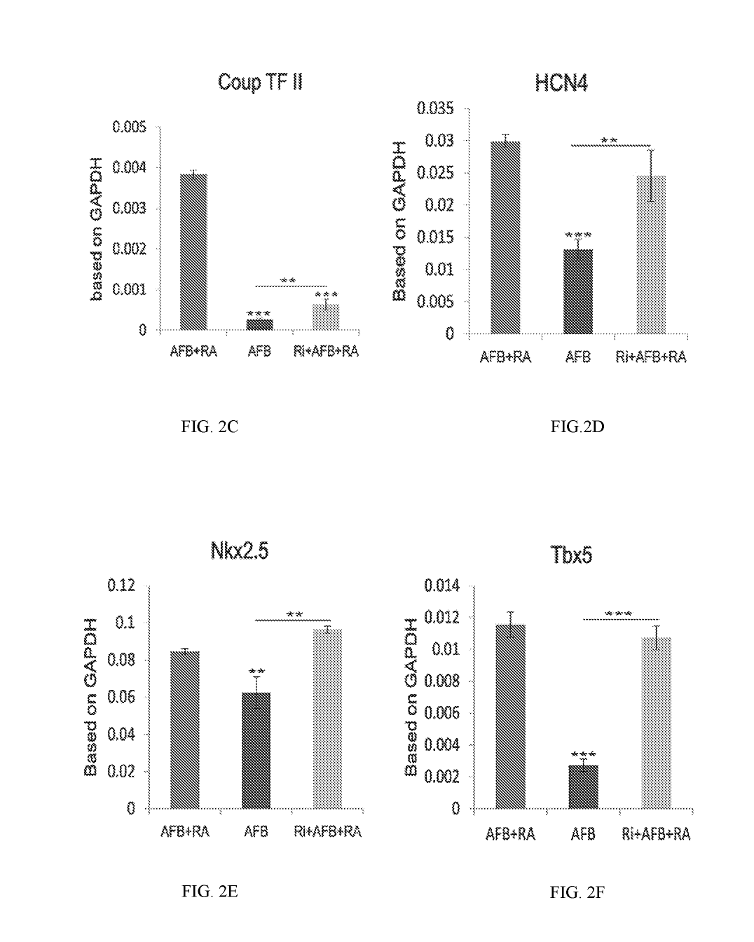

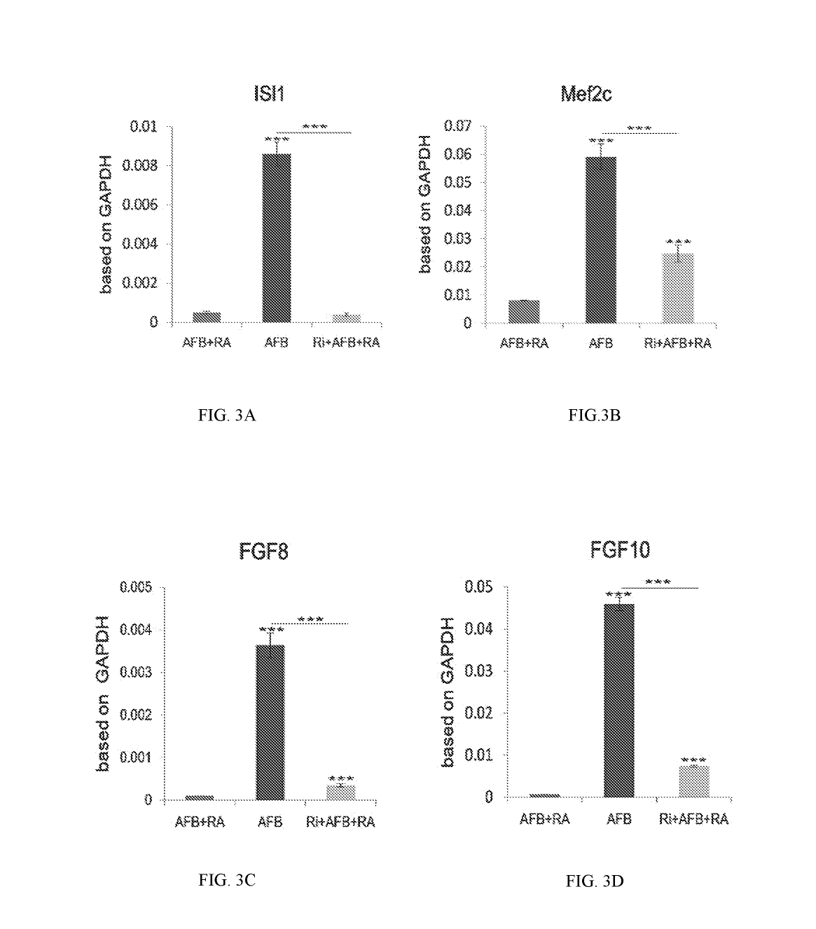

### DETAILED DISCUSSION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise.

Activin A is also known as Inhibin Beta A Chain. The Homo sapiens reference protein can be found on the NCBI national database with accession number NP 002183, version NP_002183.1. In certain embodiments, the use of functional variants, allelic variants, or active fragments are contemplated.

BMP4 is also known as bone morphogenetic protein 4. The Homo sapiens reference protein can be found on the NCBI national database with accession number NP_001193, version NP_001193.2. In certain embodiments, the use of functional variants, allelic variants, or active fragments are contemplated.

Fibroblast growth factor 2 is also known as basic fibroblast growth factor (bFGF). The Homo sapiens reference protein can be found on the NCBI national database with accession number NP_001997. version NP_001997.5. In certain embodiments, the use of functional variants, allelic variants, or active fragments are contemplated.

Noggin binds and inactivates members of the transforming growth factor-beta (TGF-beta) superfamily signaling proteins. The Homo sapiens reference protein can be found on the NCBI national database with accession number NP_005441, version NP_005441.1. In certain embodiments, the use of functional variants, allelic variants, or active fragments are contemplated.

The term “retinoic acid” refers to 3,7-dimethyl-9-(2,6,6-trimethylcyclohexen-1-yl)nona-2,4,6,8-tetraenoic acid or salts thereof. An “inhibitor of retinoic acid signaling” refers to a molecule that is an antagonist of a retinoic acid receptor (RAR) (e.g., RARα, RARβ and RARγ). The inhibitors are typically derivatives of retinoic acids such as 4-[2-[5,6-Dihydro-5,5-dimethyl-8-(4-methylphenyl)-2-naphthalenyl]ethynyl]benzoic acid (AGN193109), 4-[[[5,6-Dihydro-5,5-dimethyl-8-(3-quinolinyl)-2-naphthalenyl]carbonyl]amino]benzoic acid (BMS 195614), 4-[(1E)-2-[5,6-Dihydro-5,5-dimethyl-8-(2-phenylethynyl)-2-naphthalenyl]ethenyl]benzoic acid (BMS 493), 4-[6-[(2-Methoxyethoxy)methoxy]-7-tricyclo[3.3.1.13,7]dec-1-yl-2-naphthalenyl)benzoic acid (CD2665), 4-[5-[8-(1-Methylethyl)-4-phenyl-2-quinolinyl]-1H-pyrrolo-2-benzoic acid (ER50891), 4-(7,8,9,10-Tetrahydro-5,7,7,10,10-pentamethyl-5H-benzo[e]naphtho[2,3-b][1,4]diazepin-13-yl)benzoic acid (LE135), 6-[2-(5,6,7,8-Tetrahydro-5,5,8,8-tetramethyl-2-naphthalenyl)-1,3-dithiolan-2-yl]-2-naphthalenecarboxylic acid (MM11253), and 4-[(1E)-2-(5,6-Dihydro-5,5-dimethyl-8-phenyl-2-naphthalenyl)ethenyl]-benzoic acid (BMS 453), N-(4-Hydroxyphenyl)retinamide (Fenretinide), alkyl esters, salts, and derivatives thereof.

In certain embodiments, this disclosure contemplates that methods disclosed herein that utilize retinoic acid may be accomplished alternatively or additionally using retinoic acid receptor agonists such as alkyl esters of retinoic acid, salts and derivatives thereof. Examples include retinoic acid receptor agonists 13-cis-Retinoic acid (isotretinoin), 6-[2-(3,4-Dihydro-4,4-dimethyl-2H-1-benzothiopyran-6-yl)ethynyl]-3-pyridinecarboxylic acid ethyl ester (Tazarotene), 4-[(E)-2-(5,6,7,8-Tetrahydro-5,5,8,8-tetramethyl-2-naphthalenyl)-1-propenyl]benzoic acid (TTNPB), 4-[4-(2-Butoxyethoxy-)-5-methyl-2-thiazolyl]-2-fluorobenzoic acid (AC261066), 4′-Octyl-[1,1′-biphenyl]-4-carboxylic acid (AC55649), 6-(4-Methoxy-3-tricyclo[3.3.1.13,7]dec-1-ylphenyl)-2-naphthalenecarboxylic acid (Adapalene), 4-[(5,6,7,8-Tetrahydro-5,5,8,8-tetramethyl-2-naphthalenyl)carboxamido]benzoic acid (AM580), 4-[[(5,6,7,8-Tetrahydro-5,5,8,8-tetramethyl-2-naphthalenyl)amino]carbonyl]benzoic acid (AM80), 4-[[(2,3-Dihydro-1,1,3,3-tetramethyl-2-oxo-1H-inden-5-yl)carbonyl]amino]benzoic acid (BMS753), 3-Fluoro-4-[[2-hydroxy-2-(5,5,8,8-tetramethyl-5,6,7,8-tetrahydro-2-naphthalenyl)acetyl]amino]-benzoic acid (BMS 961), 4-(6-Hydroxy-7-tricyclo[3.3.1.13,7]dec-1-yl-2-naphthalenyl)benzoic acid (CD1530), 5-(5,6,7,8-Tetrahydro-5,5,8,8-tetramethyl-2-anthracenyl)-3-thiophenecarboxylic acid (CD2314), 6-(4-Hydroxy-3-tricyclo[3.3.1.13,7]dec-1-ylphenyl)-2-naphthalene carboxylic acid (CD437), 4-[(1E)-3-[3,5-bis(1,1-Dimethylethyl)phenyl]-3-oxo-1-propenyl]benzoic acid (CH55), 4-[(1E)-2-(5,6-Dihydro-5,5-dimethyl-8-phenyl-2-naphthalenyl)ethenyl]-benzoic acid (EC19), 4-[2-(5,6,7,8-Tetrahydro-5,5,8,8-tetramethyl-2-naphthalenyl)ethynyl)-benzoic acid (EC23), alkyl esters, salts, and derivatives thereof.

In certain embodiments, the disclosure contemplates using retinoic acid in methods disclosed herein in combination with an inhibitor of retinoic acid metabolism such as 5-[(3-Chlorophenyl)-1H-imidazol-1-ylmethyl]-1H-benzimidazole dihydrochloride (Liarozole), salts, and derivatives thereof.

Wnt signaling can be inhibited by several antagonists that bind either to the Wnt ligand itself, or to Wnt receptors. Known antagonists of Wnt signaling include Dickkopf (Dkk) proteins, Wnt Inhibitory Factor-1 (WIF-1), and secreted Frizzled-Related Proteins (sFRPs), and antibodies that bind Wnt. Other examples include 4-(2-Methyl-4-pyridinyl)-N-[4-(3-pyridinyl)phenyl]benzeneacetamide (Wnt-059), 4-[4-(4-Methoxyphenyl)-5-[[[3-(4-methylphenyl)-1,2,4-oxadiazol-5-yl]methyl]thio]-4H-1,2,4-triazol-3-yl]-pyridine (JW74), N-[(1,1-dimethylethoxy)carbonyl]-L-alanyl-(2S)-2-hydroxy-3-methylbutanoyl-L-alanine-(1S)-1-carboxy-2-methylpropyl ester (NSC668036), Trispiro[3H-indole-3,2′-[1,3]dioxane-2″,3′″-[3H]indole]-2,2′″(1H,1′″H)-dione (JW67), 3,5,7,8-Tetrahydro-2-[4-(trifluoromethyl)phenyl]-4H-thiopyrano[4,3-d]pyrimidin-4-one (XAV), N-(6-Chloro-2-benzothiazolyl)-3,4-dimethoxybenzenepropanamide (KY02111), N-(6-Methyl-2-benzothiazolyl)-2-[(3,4,6,7-tetrahydro-4-oxo-3-phenylthieno[3,2-d]pyrimidin-2-yl)thio]-acetamide (IWP2), 2-Phenoxybenzoic acid-[(5-methyl-2-furanyl)methylene]hydrazide (PNU74654), [(3aR*,4S*,7R*,7aS)-1,3,3a,4,7,7a-Hexahydro-1,3-dioxo-4,7-methano-2H-isoindol-2-yl]-N-8-quinolinylbenzamide (endo-IWR1), 4-[(3 aR,4R,7 S,7aS-rel)-1,3,3a,4,7,7a-Hexahydro-1,3-dioxo-4,7-methano-2H-isoindol-2-yl]-N-8-quinolinylbenzamide (exo-IWR1), N-(6-Methyl-2-benzothiazolyl)-2-[(3,4,6,7-tetrahydro-3-(2-methoxyphenyl)-4-oxothieno[3,2-d]pyrimidin-2-yl)thio]-acetamide (IWP4), 2,5-Dichloro-N-(2-methyl-4-nitrophenyl)benzenesulfonamide (FH535), 6-Cyclohexyl-3-(2-furanyl)-1,2,4-triazolo[3,4-b][1,3,4]thiadiazole (Cardionogen1), 5-[[2,5-Dimethyl-1-(3-pyridinyl)-1H-pyrrol-3-yl]methylene]-3-phenyl-2,4-thiazolidinedion (iCRT14), 2-[3-[[4-(4-Methoxyphenyl)-5-(4-pyridinyl)-4H-1,2,4-triazol-3-yl]thio]propyl]-1H-benz[de]isoquinoline-1,3(2H)-dione (WIKI4), N-[4-[2-Ethyl-4-(3-methylphenyl)-5-thiazolyl]-2-pyridinyl]benzamide (TAK715), N-[4-[[[[Tetrahydro-4-(4-methoxyphenyl)-2H-pyran-4-yl]methyl]amino]carbonyl]phenyl]-2-furancarboxamide (JW55), N-(5-Phenyl-2-pyridinyl)-2-[(3,4,6,7-tetrahydro-4-oxo-3-phenylthieno[3,2-d]pyrimidin-2-yl)thio]acetamide (IWPL6), 2-[4-(1-Methyl ethyl)phenyl]-4H-1-benzopyran-4-one (MN64), N-(6-Methyl-2-benzothiazolyl)-2-[(3,4,6,7-tetrahydro-3,6-dimethyl-4-oxothieno[3,2-d]pyrimidin-2-yl)thio]acetamide (IWP12), rel-2-[4-[6-[(3R,5S)-3,5-Dimethyl-1-piperazinyl]-4-methyl-3-pyridinyl]phenyl]-3,7-dihydro-7-methyl-4H-pyrrolo[2,3-d]pyrimidin-4-one (AZ6102), alkyl esters, salts, and derivatives thereof.

As used herein, the term “derivative” refers to a structurally similar compound that retains sufficient functional attributes of the identified analogue. The derivative may be structurally similar because it is lacking one or more atoms, e.g., replacing an amino group, hydroxyl, or thiol group with a hydrogen, substituted, a salt, in different hydration/oxidation states, or because one or more atoms within the molecule are switched, such as, but not limited to, replacing a oxygen atom with a sulfur atom or replacing an amino group with a hydroxyl group. The derivative may be a prodrug, comprise a lipid, polyethylene glycol, saccharide, polysaccharide. A derivative may compound disclosed herein substituted with one or more substituents. Derivatives may be prepared by any variety of synthetic methods or appropriate adaptations presented in synthetic or organic chemistry textbooks, such as those provide in March's Advanced Organic Chemistry: Reactions, Mechanisms, and Structure, Wiley, 6th Edition (2007) Michael B. Smith or Domino Reactions in Organic Synthesis, Wiley (2006) Lutz F. Tietze hereby incorporated by reference.

The term “substituted” refers to a molecule wherein at least one hydrogen atom is replaced with a substituent. When substituted, one or more of the groups are “substituents.” The molecule may be multiply substituted. In the case of an oxo substituent (“═O”), two hydrogen atoms are replaced. Example substituents within this context may include halogen, hydroxy, alkyl, alkoxy, nitro, cyano, oxo, carbocyclyl, carbocycloalkyl, heterocarbocyclyl, heterocarbocycloalkyl, aryl, arylalkyl, heteroaryl, heteroarylalkyl, —NRaRb, —NRaC(═O)Rb, —NRaC(═O)NRaNRb, —NRaC(═O)ORb, —NRaSO2Rb, —C(═O)Ra, —C(═O)ORa, —C(═O)NRaRb, —OC(═O)NRaRb, —ORa, —SRa, —SORa, —S(═O)₂Ra, —OS(═O)₂Ra and —S(═O)₂ORa. Ra and Rb in this context may be the same or different and independently hydrogen, halogen hydroxyl, alkyl, alkoxy, alkyl, amino, alkylamino, dialkylamino, carbocyclyl, carbocycloalkyl, heterocarbocyclyl, heterocarbocycloalkyl, aryl, arylalkyl, heteroaryl, and heteroarylalkyl. The substituents may further optionally be substituted.

Variants of polypeptides may include 1 or 2 amino acid substitutions or conserved substitutions. Variants may include 3 or 4 amino acid substitutions or conserved substitutions. Variants may include 5 or 6 or more amino acid substitutions or conserved substitutions. Variant include those wherein not more than 1% or 2% of the amino acids are substituted. Variant include those wherein not more than 3% or 4% of the amino acids are substituted. Variants include proteins with greater than 80%, 89%, 90%, 95%, 98%, or 99% identity or similarity. Variants can be tested by mutating a vector to produce appropriate codon alternatives for polypeptide translation. Active variants and fragments can be identified with a high probability using computer modeling. Shihab et al. report an online genome tolerance browser. BMC Bioinformatics. 2017, 18(1):20. Ng et al. report methods of predicting the effects of amino acid substitutions on protein function. Annu Rev Genomics Hum Genet. 2006, 7:61-80. Teng et al. Approaches and resources for prediction of the effects of non-synonymous single nucleotide polymorphism on protein function and interactions. Curr Pharm Biotechnol. 2008, 9(2):123-33.

In certain embodiments, sequence “identity” refers to the number of exactly matching amino acids (expressed as a percentage) in a sequence alignment between two sequences of the alignment calculated using the number of identical positions divided by the greater of the shortest sequence or the number of equivalent positions excluding overhangs wherein internal gaps are counted as an equivalent position. For example, the polypeptides GGGGGG and GGGGT have a sequence identity of 4 out of 5 or 80%. For example, the polypeptides GGGPPP and GGGAPPP have a sequence identity of 6 out of 7 or 85%. In certain embodiments, any recitation of sequence identity expressed herein may be substituted for sequence similarity. Percent “similarity” is used to quantify the similarity between two sequences of the alignment. This method is identical to determining the identity except that certain amino acids do not have to be identical to have a match. Amino acids are classified as matches if they are among a group with similar properties according to the following amino acid groups: Aromatic—F Y W; hydrophobic-A V I L; Charged positive: R K H; Charged negative—D E; Polar—S T N Q. The amino acid groups are also considered conserved substitutions.

As used herein, the terms “fragment”, “functional fragment” or similar terms shall be given their ordinary meaning and shall refer to a portion of an amino acid sequence (or polynucleotide encoding that sequence) that has at least about 70%, preferably at least about 80%, more preferably at least about 90%, 95%, 96%, 97%, 98% or 99% of the function of the corresponding full-length amino acid sequence (or polynucleotide encoding that sequence). Methods of detecting and quantifying functionality of such fragments are established in the art.

As used herein a “stem cell” refers to a cell, under certain physiologic or experimental conditions, that can be induced to become tissue- or organ-specific cells with special functions. Stem cell types include embryonic stem cells, adult stem cells, and induced pluripotent stem cells. An adult stem cell or somatic cell is found among differentiated cells in a tissue or organ and can renew itself. Adult stem cells can differentiate to yield some or all of the major specialized cell types of the tissue or organ. Examples of adult stem cells include MSCs. Induced pluripotent stem cells are cells that have been naturally differentiated but exposed to chemicals and/or biologic materials in vitro (treated with reprogramming factors) that allow the cell to differentiate into a larger capacity of specialized cells.

The term “mesenchymal stromal cells” refers to the subpopulation of fibroblast or fibroblast-like nonhematopoietic cells with properties of plastic adherence and capable of in vitro differentiation into cells of mesodermal origin which may be derived from bone marrow, adipose tissue, umbilical cord (Wharton's jelly), umbilical cord perivascular cells, umbilical cord blood, amniotic fluid, placenta, skin, dental pulp, breast milk, and synovial membrane, e.g., fibroblasts or fibroblast-like cells with a clonogenic capacity that can differentiate into several cells of mesodermal origin, such as adipocytes, osteoblasts, chondrocytes, skeletal myocytes, or visceral stromal cells. The term, “mesenchymal stem cells” refers to the cultured (self-renewed) progeny of primary mesenchymal stromal cell populations.

Bone marrow derived mesenchymal stromal cells are typically expanded ex vivo from bone marrow aspirates to confluence. Certain mesenchymal stromal/stem cells share a similar set of core markers and properties. Certain mesenchymal stromal/stem cells may be defined as positive for CD105, CD73, and CD90 and negative or low for CD45, CD34, CD14, and have the ability to adhere to plastic. See Dominici et al. Minimal criteria for defining multipotent mesenchymal stromal cells. The International Society for Cellular Therapy position statement. Cytotherapy, 2006, 8(4):315-7.

Adipose tissue-derived multipotent stem cells (ADMSCs) are multipotent, undifferentiated, self-renewing progenitor cell population isolated from adipose tissue. One method to isolate ADSCs from fat tissue relies on a collagenase digestion, followed by centrifugal density gradient separation. In vitro, ADMSCs typically display a spindle-shaped morphology and lack the intracellular lipid droplets as seen in adipocytes. Isolated ADMSCs are typically expanded in monolayer cultures with a growth medium containing fetal bovine serum and/or human platelet lysate. ADMSCs have the stem cell-specific surface markers, such as CD90, CD105, CD73, and lack the expression of the hematopoietic markers CD45 and CD34.

As used herein, and unless the context clearly suggests otherwise, the term “MSCs” refers to multipotent stem cells such as mesenchymal stromal cells, mesenchymal stem cells, and adipose tissue-derived multipotent stem cells.

As used herein a “growth medium” or “media” refers to a composition that contains components, such as vitamins, amino acids, inorganic salts, a buffer, and a fuel, e.g., acetate, succinate, and/or a saccharide, that support the growth and maintenance of cell lines. Components in the growth medium may be derived from blood serum or the growth medium may be serum-free. The growth medium may optionally be supplemented with albumin, lipids, insulin and/or zinc, transferrin or iron, selenium, ascorbic acid, and an antioxidant such as glutathione, 2-mercaptoethanol or 1-thioglycerol. Media may contain a reducing agent such as glutathione. Media may contain biotin, vitamin B12, and PABA. In addition, media may contain inositol and choline. Medium may or may not be supplemented with serum, e.g., 1-5% or 5-10% Fetal Bovine Serum (FBS). Media may use a sodium bicarbonate buffer system (2.0 g/L). Other contemplated components in these growth medium include ascorbic acid, L-alanine, zinc sulfate, human transferrin, albumin, insulin, ammonium metavanadate, cupric sulfate, manganous chloride, sodium selenite, ethanolamine, and sodium pyruvate.

Retinoic Acid Enriches Left Ventricular-Like Cells Derivation from Human Embryonic Stem Cells

Conversion of human embryonic stem cells (hESCs) is model for studying heart development. A major problem with their translational use is that differentiation of hESCs using current methods yield random mixtures of atrial, ventricular and nodal-like cardiac myocytes. Experiments were performed to test whether stage-specific manipulation of RA signaling may enable cardiac derivation of hESCs toward the left ventricular myocytes.

Human ESCs (H9) were differentiated to cardiac lineage by treatment with Activin A, BMP4 and bFGF (ABF, control group) for two days (day 1-2) Mesoderm formation was identified by brachyury (T)+ cells, and the ensuing pan-cardiac myocyte lineage was marked by cTnT+ cells. In control, this process was highly efficient, reaching 75% and 82%, respectively. Early inhibition of RA with a pan-RA receptor antagonist, BMS-189493, increased mesoderm induction, but the majority (86%) of the mesodermal cells failed to become cardiac myocytes. Early activation of RA failed to generate mesoderm lineage. In contrast, late (day 6-9) activation of RA (Late-RA) led to >90% cTnT+ population. Importantly, ventricular markers, IRX4 and HRT2, were up-regulated, while an atrial marker, CoupTFII, was down-regulated in the Late-RA cardiomyocytes compared to control. Furthermore, the Late-RA cardiomyocytes expressed higher transcript and protein levels of the first heart field (FHF) markers, NKX2.5, TBX5 and HCN4.

Conversely, the second heart field (SHF) markers, ISL1, FGF8 and FGF10 were downregulated in the Late-RA cardiomyocytes compared to control. Taken together, the data indicate that late-stage activation of the RA signaling enriches cardiac myocytes with gene expression profile of the FHF cells that populate the majority of the left ventricle. Reported are specific and robust derivation of the left ventricular-like cells by activating RA signaling during the late stage of hESC differentiation.

### EXAMPLES

### AFB Protocol

To create the cardio-like cells from hESCs, at the early stage (day 0-3), hESCs were treated with ActivinA, bFGF and BMP4 to induce mesoderm and cardiac mesoderm cells generation. Noggin treatment expanded more cardiac lineage cells during (day 3-5). Wnt inhibitor promoted cardiac progenitor cells into cardio-like cells during day 5-11.

### AFB+RA Protocol

To create atrial-like cells from hESCs, at the early stage (day 0-3), hESCs were treated with ActivinA, bFGF and BMP4 to induce mesoderm and cardiac mesoderm cells generation. Noggin treatment expanded more cardiac lineage cells during day 3-5. Wnt inhibitor promoted differentiation of cardiac progenitor cells into cardio-like cells during day 5-11. The treatment of RA during day 5-8 specified atria-like subtypes.

### Ri+AFB+RA Protocol

To create first heart field (FHF) derived left ventricular-like cells from hESCs, at the early stage (day 0-3), hESCs were treated with ActivinA, bFGF, BMP4 and additional short time (day 0-1) treatment of BMS4.93(Ri) to induce mesoderm and cardiac mesoderm cells generation. Noggin treatment expanded more cardiac lineage cells during day 3-5. Wnt inhibitor promoted cardiac progenitor cells into cardiomyocytes during day 5-11. The treatment of RA during day 5-8 specified first heart field (FHF) derived left ventricular-like cell subtypes.

1. A method of making left ventricular-like cells comprising:
a) mixing stem cells with Activin A, BMP4, bFGF, and an inhibitor of retinoic acid signaling providing inhibitor treated dividing cells;
b) mixing the inhibitor treated dividing cells with noggin providing noggin treated cells; and
c) mixing the noggin treated cells with retinoic acid and a Wnt inhibitor providing left ventricular-like cells.

2. The method of claim 1 wherein the ventricular-like cells are positive for Nkx2.5 are positive for IRX4, are positive for HRT2, and are negative for ISL1.

3. The method of claim 1 wherein the inhibitor treated dividing cells are exposed to the inhibitor of retinoic acid signaling for not more than one day.

4. The method of claim 1 wherein the inhibitor treated dividing cells are exposed to Activin A, BMP4, and bFGF for not more than three days.

5. The method of claim 1 wherein the noggin treated cells are exposed to noggin for not more than two days.

6. The method of claim 1 wherein mixing the noggin treated cells with retinoic acid is started five days after mixing stem cells with Activin A, BMP4, bFGF, and an inhibitor of retinoic acid signaling.

7. The method of claim 1 wherein the noggin treated cells are exposed to retinoic acid for not more than three days.

8. The method of claim 1 wherein mixing the noggin treated cells with a Wnt inhibitor is started five days after mixing stem cells with Activin A, BMP4, bFGF, and an inhibitor of retinoic acid signaling.

9. A method of making atrial-like cells comprising:
a) mixing stem cells with Activin A, BMP4, and bFGF providing treated dividing cells;
b) mixing the treated dividing cells with noggin providing noggin treated cells; and
c) mixing the noggin treated cells with retinoic acid and a Wnt inhibitor providing atrial-like cells.

10. The method of claim 9 wherein the atrial-like cells are positive for Nkx2.5, are positive for Coup TF II, are negative for IRX4, and are negative for ISL1.

11. The method of claim 9 wherein the treated dividing cells are exposed to Activin A, BMP4, and bFGF for not more than three days.

12. The method of claim 9 wherein the noggin treated cells are exposed to noggin for not more than two days.

13. The method of claim 9 wherein mixing the noggin treated cells with retinoic acid is started five days after mixing stem cells with Activin A, BMP4, and bFGF.

14. The method of claim 9 wherein the noggin treated cells are exposed to retinoic acid for not more than three days.

15. The method of claim 9 wherein mixing the noggin treated cells with a Wnt inhibitor is started five days after mixing stem cells with Activin A, BMP4, and bFGF.

16. A method of making ventricular-like cells comprising:
a) mixing stem cells with Activin A, BMP4, and bFGF providing treated dividing cells;
b) mixing the treated dividing cells with noggin providing noggin treated cells; and
c) mixing the noggin treated cells with a Wnt inhibitor providing ventricular-like cells.

17. The method of claim 16 wherein the ventricular-like cells are positive for Nkx2.5, are positive for IRX4, and are positive for ISL1.

18. The method of claim 16 wherein the treated dividing cells are exposed to Activin A, BMP4, and bFGF for not more than three days.

19. The method of claim 16 wherein the noggin treated cells are exposed to noggin for not more than two days.

20. The method of claim 16 wherein mixing the noggin treated cells with a Wnt inhibitor is started five days after mixing stem cells with Activin A, BMP4, and bFGF.

21-25. (canceled)
